(19) United States
(12) Patent Application Publication
(10) Pub. No.: US 2020/0346049 A1
(43) Pub. Date: Nov. 5, 2020

(54) WAXING BASED SKINCARE TREATMENT METHOD AND SYSTEM

(71) Applicant: EWC P&T, LLC, Plano, TX (US)

(72) Inventors: Sherry Helene Baker, New York, NY (US); David Coba, Fort Lauderdale, FL (US); Renee Ordino, Cream Ridge, NJ (US); Joshua Coba, Aventura, FL (US)

(21) Appl. No.: 16/519,717

(22) Filed: Jul. 23, 2019

Related U.S. Application Data
(60) Provisional application No. 62/842,820, filed on May 3, 2019.

(51) Int. Cl. A61Q 7/02; A61K 8/02; A61Q 19/00

(52) U.S. Cl. CPC A61Q 7/02; A61K 2800/59; A61Q 19/00; A61K 8/0208

(57) ABSTRACT

A heat amplified skin treatment method and system including applying a predetermined one of a plurality of serum solutions to the skin. A wax composition is pre-heated into a liquid state and applied to the skin in covering relation to the applied, predetermined serum solution and in heat transferring relation to the area of the skin. A mask forms, via a cooling of the wax composition, and is disposed in encapsulating relation to the predetermined serum solution, on the skin. After expiration of a predetermined time period, the mask is removed. The results of the skin treatment are further enhanced by the application of selected serum-locking, cleanser and toner compositions at different stages during the skin treatment.

### CLAIM OF PRIORITY

The present Non-Provisional patent application claims priority pursuant to 35 U.S.C. Section 119(e) to a currently pending and prior filed Provisional patent application, namely, that having Ser. No. 62/842,820 filed on May 3, 2019, the content of which is incorporated herein by reference in its entirety.

### BACKGROUND OF THE INVENTION

### Field of the Invention

The present invention is directed to a system and attendant method of use and application of a therapeutic, heat amplified skincare treatment. A predetermined one of a possible plurality of task specific “boosters” or serum solutions is applied to the user's skin and subsequently “encapsulated” by a pre-heated wax composition. Heat from the wax composition facilitates penetration and/or infusion of therapeutic ingredients of the applied serum solution, into the skin of the user.

### Description of the Related Art

The therapeutic and/or cosmetic skincare industry enjoys a significant degree of popularity in the United States and most advanced/industrialized countries throughout the world. A relatively recent segment of this industry is dedicated to facial treatments which can be accomplished in a relatively short amount of time, such as generally under 30 minutes. This so-called “fast facial” procedure or technique is rapidly growing and typically involves a cleansing through an exfoliation and extraction process. Such an increase in popularity is based at least in part on the shorter time requirements for the procedure and generally lower costs. Such an increase in popularity is based at least in part on the shorter time requirements for the procedure and generally lower costs. Therefore, it would be highly beneficial to develop a unique system and method that can be accomplished in a relatively short amount of time similar to a “fast facial”, but that provides significantly improved benefits and results through heat infusion without needing to resort to the exfoliation and extraction process typically associated with that of a facial.

In more statistical terms, about 14.5 million women, between the ages of 18-59, have had a facial treatment in the United States in the past year. Of these, generally about 27% have enjoyed the “fast facial” technique. Moreover, consumers spend about $75 on average, per fast facial procedure, resulting in a category value of generally about $485 million in 2018. However, there is a recognized variance in what consumers spend in a given session, most likely because the fast facial category is not standardized and remains generally undefined.

It is generally believed that the growth of the fast facial category is influenced more by availability of service than by the need of such services. Accordingly, it is believed that the availability of the fast facial technique can increase both frequency and demand for such services. Further, the reasons consumers utilize such fast facial treatments include health goals; overall cosmetic results and the solving of undesirable skin issues. It has also been determined that fast facial consumers may prefer to get treatments which are typically not capable of being replicated in their entirety at home. Contributing factors to this preference may include the availability of other desirable procedures at commercial sites, such as a head massage, soothing hand treatment, etc. However, consumers also prioritize options for ongoing skincare such as the availability of “sample size products” which may be utilized prior to purchasing a full-size and/or “spot atmosphere” treatments.

As indicated, one of the factors in choosing fast facial treatment is cost. However investigation indicates that the desirability of achieving health/skin goals and/or improving an existing medical skin issue may override a moderate or reasonable increase in expenditures for such treatments. In more specific terms, consumers would pay an additional fee for the addition of unique, customized, specialized treatments or procedures, which may not be commonplace and/or otherwise available with a typical or conventional treatment.

Accordingly, there is an opportunity in the skincare industry for such a unique system and attendant method of implementation for a skin treatment, which may associate with and even expand on the “fast facial” and “skincare” categories. This proposed skin treatment can help overcome the disadvantages of known facial and other skincare treatment procedures.

Unique advantages associated with such an improved skin treatment would be the utilization of “heat amplification” applied to the skin by an application of a specialized preheated wax composition. An improved system and/or method would also include the “encapsulation” of the initially applied “booster” or serum solution by the wax composition, as it cools into a substantially solidified, film-like mask. Thereafter the formation of the mask, upon cooling of the preheated wax composition, will encapsulate the selected serum solution resulting in an “incubator effect”, which significantly enhances the absorption, penetration and uniquely deep infusion of the beneficial, therapeutic ingredients/properties, present in an applied booster or serum solution, each being formulated to accomplish a specific task and different therapeutic result, including anti-aging or a more youthful appearance, skin brightening or skin clarification.

Conventional facial treatments may have contemplated the use of creams, ointments, gels, etc. being applied in covering relation to an individual's skin, in somewhat of a mask-like configuration. However, such conventional treatments have been recognized in the skincare industry as being a messy, sticky and difficult to remove. Further compositions for such conventional skin treatments often require animal testing to assure safety to a human user. Also, conventional skin treatments of this type lack the ability to encapsulate the skin and provide a significant “incubator effect”, which in turn facilitates absorption of therapeutic and/or other beneficial ingredients/properties applied to the skin.

Accordingly, an improved and proposed system and method of heat amplified skin treatment overcomes these noted disadvantages by facilitating heat amplification of the skin, as well as forming an encapsulating, incubating mask from a substantially solidified, film-like wax mask. The at least partial solidification of the wax mask also facilitates its removal in substantially one piece.

Therefore, an improved and proposed skin treatment, of the type described in greater detail hereinafter could be performed, in a timely manner, at a professional or commercial on-site location. Further, additional therapeutic creams, sprays, solutions, compositions, etc. in various forms could be made available to consumers for periodic in-home use, thereby extending the intended and desired beneficial is results initially obtained at an on-site treatment facility.

### SUMMARY OF THE INVENTION

The present invention is directed to a method and system for implementation of a heat amplified skin treatment for the face and other skin areas, initially and preferably applied, on a regular periodic basis, at an on-site treatment facility. More specifically, it is believed that the cosmetic and other beneficial results of the skin treatment of the present invention may be cumulative and significantly enhanced if applied on a regular, periodic basis such as, but not limited to, every three weeks.

However, in order to further enhance and extend the resultant benefits of the skin treatment, a plurality of products, which may be initially incorporated into the treatment at the on-site location, may be made available to consumers for regular, periodic treatment at home. The “at home” or off-site application of the various supplementary applied products is further facilitated by a “waterless” application. As used herein, the term “waterless” is meant to describe that each of the plurality of products utilized in the performance of the on-site or at home application of the treatment is accomplished without the use or requirement of additional water. However, it is acknowledged that the formulation, composition, etc. of the various products may initially include some form of water or liquid.

One or more features of the skin treatment of the present invention includes the use of a plurality of “task specific” serum solutions or “boosters”. Each of the plurality of serum solutions is formulated to have different therapeutic properties from one another as well as at least partially distinctive ingredients. By way of a non-limiting example and as explained in greater detail hereinafter, the plurality of therapeutic serum solutions or “boosters” may include, but are not necessarily limited to, a youth (anti-aging) serum, a brightening serum solution and a clarifying serum solution.

In more specific terms, the youth serum solution is primarily formulated to improve the appearance of fine lines and wrinkles; enhance skin texture and evenness as well as improve skin firmness and elasticity. The brightening serum solution is primarily formulated to improve skin texture and radiance as well as skin evenness and skin tone. Concurrently hydration of the skin is also a beneficial result. The clarifying serum solution is primarily formulated to clear, calm, soothe and balance the skin to the extent of decreasing the oil (sebum) content, and decrease pore size, while also improving skin moisture.

It should be recognized that “task specific” formulations of the plurality of serum solutions or boosters is intended to meet the demands and desires of different consumers which may vary according to age, body application and condition of the skin or existing skin medical/cosmetic factors, such as acne, etc. Further by way of a non-limiting example, a younger participant or user would most probably not need the therapeutic benefits associated with the youth serum solution. Accordingly, the other brightening or clarifying boosters may be more appropriate. Therefore, application of the heat amplified skin treatment would involve the utilization of a predetermined one of the plurality of serum solutions or boosters, most appropriate or desired by an individual customer/user.

Further, one feature incorporated in the method and system of the present invention is the application of heat to the skin of the user. Such an “amplified heat” feature accomplishes a deeper penetration, infusion and/or absorption of the beneficial, therapeutic, active ingredients/properties of the applied serum solution. Moreover, heat amplification occurs after the selected serum solution has been applied and remains on the user's skin.

Therefore, one feature of the method and system of the heat amplified skin treatment of the present invention comprises the application of a pre-heated wax composition to the skin in overlying, covering relation to the selected, predetermined serum solution. The wax composition is pre-heated to a substantially liquid state which allows the heated wax composition to be easily applied to the skin with a brush or other appropriate applicator. When so applied, the wax composition is disposed and formulated to substantially “encapsulate” the predetermined, selected serum solution. Moreover, the temperature of the pre-heated wax is raised to a level (generally about 135° F.) that initially maintains the wax composition in a liquid state. Further by way of example, the wax composition may be applied in a substantially liquid state at a somewhat lower temperature of generally about 122° F.-124° F., thereby providing an overall comforting feeling to the skin.

While preferred, the precise temperature of the pre-heated wax composition, at the time of application to the skin, is not necessarily limited to these precise temperature parameters. Determinative factors should include a temperature of the wax composition sufficient to facilitate heat transfer to the skin of the user, while “encapsulating” the predetermined serum solution and thereby applying an “incubator affect” to the underlying predetermined serum solution. However, the temperature of the wax composition should obviously be sufficiently low to avoid discomfort to the skin. Once applied, in the manner generally set forth above, the wax composition is allowed to cool and thereby form a substantially solidified film-like mask. As indicated the mask is disposed in overlying, encapsulating relation to the previously applied selected, predetermined serum solution.

Therefore, as implemented, the method and system of the heat amplified skin treatment of the present invention comprises the application of a predetermined one of the plurality of serum solutions or boosters, followed by the application of the pre-heated wax and subsequent formation of an encapsulating, incubating mask, as set forth above. However, in addition, the method and system of the present invention includes additional treatments/techniques utilizing specialized solutions, compositions, etc. to further enhance the intended therapeutic and cosmetic benefits for the skin.

In more specific terms, prior to application of the selected, predetermined serum solution, the skin is cleansed by applying a purifying cleansing lotion preferably by utilizing one or more micellar wipes. Thereafter, a moisturizing toner is applied to the skin, preferably in mist form, thereby facilitating moistening the skin and possibly imparting a relaxing aromatic sent. In at least one embodiment, a serum locking composition is applied in overlying, covering relation to the selected, predetermined serum solution. The serum locking composition is thoroughly and manually “worked-in” to the previously applied predetermined serum solution in order to facilitate an adhering, encapsulating disposition of the wax composition to the skin. As such, a roller or other appropriate applicator instrument may be utilized to assure proper infusion of the serum locking composition.

Yet additional features of the present invention associated with the implementation of the heat amplified skin may include a manual pressing or “tapping” of the wax into the skin. Such a manual pressing or tapping facilitates the aforementioned encapsulation of the wax composition and mask of the underlying, predetermined serum solution and serum locking composition. Such manual pressing or “tapping” can be accomplished by the hands/fingers of an individual implementing the heat amplified skin treatment.

After a predetermined time period and subsequent to the cooling of the pre-heated wax composition into a substantially solidified mask, the mask is removed and one or more micellar wipes are utilized to thoroughly cleanse the skin. This cleansing procedure is specifically intended to remove any remaining residue of the wax composition, formed mask, predetermined serum solution and serum locking composition. Subsequently, a micro-mist toner solution is then applied, preferably in mist form, in order to lightly moisten the skin and possibly impart a relaxing aromatic sent.

As set forth above and as described in greater detail hereinafter, additional therapeutic creams, sprays, solutions and compositions in various forms could be made available to consumers for periodic in-home or off-site use, in order to extend the intended and desired results initially obtained at an on-site treatment facility.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a schematic presentation of one embodiment of the heat amplified skin treatment method and system of the present invention for the face.

FIGS. 2A-2B is a schematic presentation collectively representative of the method and system of the present invention of the heat amplified skin treatment for the face including one embodiment of an implementation thereof.

FIG. 3 is a perspective view of one embodiment of an applicator instrument which may be utilized in implementing a portion of the present invention, as represented in FIGS. 2A-2B.

FIG. 4 is a plan view of one embodiment of an eye cover, prior to being applied and which may be associated with a portion of the implementation of the present invention as represented in the embodiment of FIGS. 2A-2B.

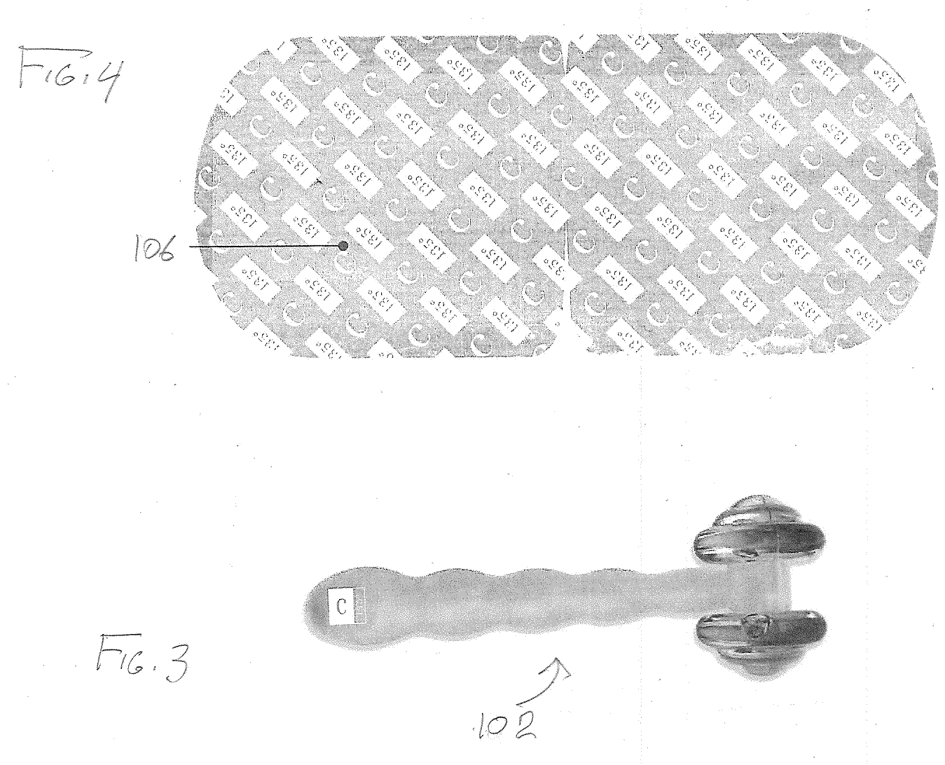

Like reference numerals refer to like parts throughout the several views of the drawings.

### DETAILED DESCRIPTION OF THE PREFERRED EMBODIMENT

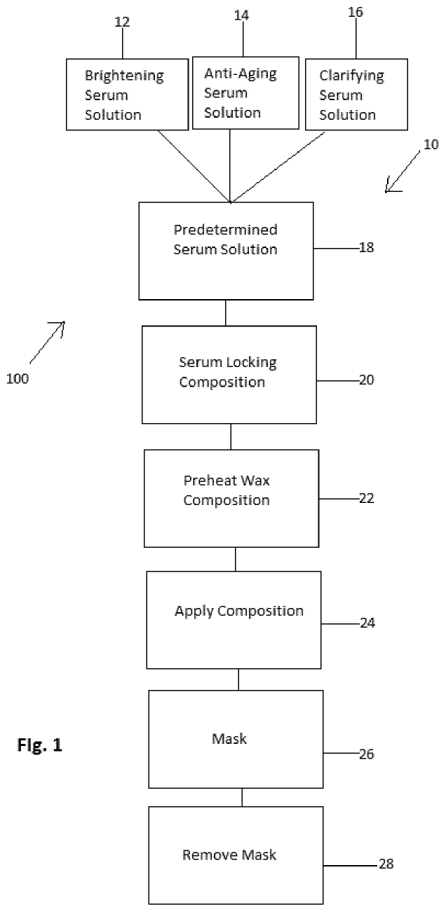

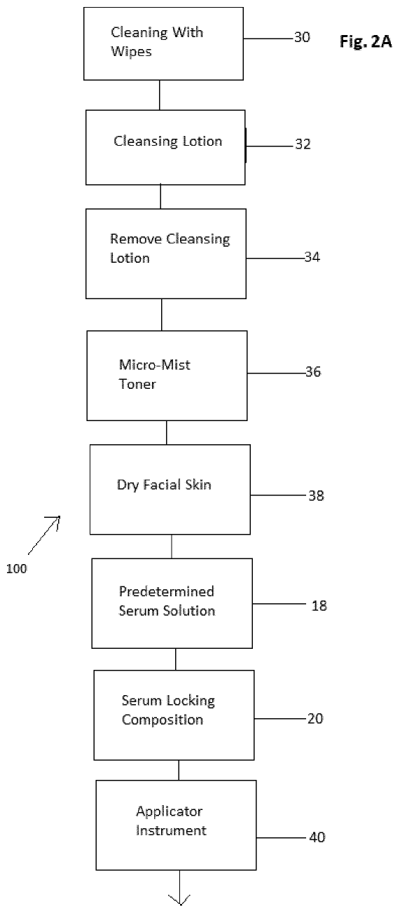

The method and system of the heat amplified skin treatment of the present invention for the face is set forth in detail with reference to the accompanying Figures and are collectively and generally indicated as 100 in FIGS. 1-2B. Moreover, as represented in FIG. 1, one preferred embodiment of the present invention is generally indicated as 10, which schematically emphasizes the “heat amplification” characteristics of the skin treatment. Accordingly, the method and system of the skin treatment 100 provides for a selection of a predetermined one of a plurality of serum solutions boosters 12, 14 and 16. Each of the plurality of serum solutions is specifically tasked to provide different cosmetic/therapeutic benefits to the skin. As such, each of the plurality of serum solutions or boosters 12, 14 and 16 includes different therapeutic properties and for the most part, different therapeutic ingredients. The plurality of specifically tasked serum solutions or boosters include a brightening serum solution 12, formulated to improve skin texture, skin radiance, skin evenness and skin tone as well as skin hydration. As a result, the brightening serum solution 12 is intended to physically improve skin tone and appearance. As such, the therapeutic ingredients formulated to define the brightening serum solution 12 may include:

### Ingredients: Brightening Serum Solution

WATER (EAU/AQUA), NEOPENTYL GLYCOL DICAPRYLATE/DICAPRATE, CAPRYLIC/CAPRIC TRIGLYCERIDE, DIMETHICONE, PROPANEDIOL, ASCORBYL GLUCOSIDE, C12-13 ALKYL LACTATE, SUCROSE DISTEARATE, AMMONIUM ACRYLOYLDIMETHYLTAURATE/VP COPOLYMER, STEARETH-10, LAMINARIA OCHROLEUCA EXTRACT, PHENOXYETHANOL, PENTYLENE GLYCOL, PALMITOYL PROLINE, FRAGRANCE (PARFUM), SODIUM CITRATE, SODIUM STEAROYL GLUTAMATE, CHLORPHENESIN, PAPAIN, SODIUM HYDROXIDE, SODIUM GLUCONATE, XANTHAN GUM, T-BUTYL ALCOHOL, MAGNESIUM PALMITOYL GLUTAMATE, SODIUM PALMITOYL SARCOSINATE, ETHYLHEXYLGLYCERIN, BUTETH-3, 1,2-HEXANEDIOL, CAPRYLYL GLYCOL, CARBOMER, SODIUM BENZOTRIAZOLYL BUTYLPHENOL SULFONATE, CITRIC ACID, ALCOHOL, ALGIN, TRIS (TETRAMETHYLHYDROXYPIPERIDINOL) CITRATE, TOCOPHEROL, TRIBUTYL CITRATE

As also represented in FIG. 1, the plurality of serum solutions or boosters may also include A youth (anti-aging) serum solution 14, formulated to improve and/or reduce the appearance of fine lines and wrinkles and help diminish spots an uneven skin tone. Further the youth serum solution 14 promotes skin evenness, firmness and elasticity, while improving the overall hydration of the skin. As such, the therapeutic ingredients formulated to define the brightening serum solution 14 may include:

### Ingredients: Youth Serum Solution

WATER (EAU/AQUA), DICAPRYLYL CARBONATE, GLYCERETH-26, GLYCERIN, PROPANEDIOL, LAUROYL LYSINE, ALOE BARBADENSIS LEAF JUICE, PALMITOYL PROLINE, HYDROLYZED COLLAGEN, SOLUBLE COLLAGEN, C14-22 ALCOHOLS, PHENOXYETHANOL, PENTYLENE GLYCOL, POLYACRYLATE-13, HYDROXYETHYL ACRYLATE/SODIUM ACRYLOYLDIMETHYL TAURATE COPOLYMER, CHLORPHENESIN, POLYISOBUTENE, C12-20 ALKYL GLUCOSIDE, FRAGRANCE (PARFUM), POLYACRYLATE CROSSPOLYMER-6, MAGNESIUM PALMITOYL GLUTAMATE, SODIUM PALMITOYL SARCOSINATE, ETHYLHEXYLGLYCERIN, BUTETH-3, SORBITAN ISOSTEARATE, HYDROLYZED SOYBEAN EXTRACT, POLYSORBATE 20, BIOSACCHARIDE GUM-1, SODIUM BENZOTRIAZOLYL BUTYLPHENOL SULFONATE, POLYSORBATE 60, ALCOHOL, SODIUM HYDROXIDE CARAMEL, TRIS (TETRAMETHYLHYDROXYPIPERIDINOL) CITRATE, TOCOPHEROL, BENZOIC ACID, SORBIC ACID, T-BUTYL ALCOHOL, TRIBUTYL CITRATE, YELLOW 5 (CI 19140)

The plurality of serum solutions or boosters may also include a clarifying serum solution 16 including therapeutic ingredients intended to decrease oily skin (sebum). In addition, the clarifying serum solution 16 decreases pore size, soothes and balances the skin and further improves skin moisture. As such, the therapeutic ingredients formulated to define the clarifying serum solution 16 may include:

### Ingredients: Clarifying Serum Solution

WATER (EAU/AQUA), DICAPRYLYL CARBONATE, ETHYL LINOLEATE, GLYCERIN, TRICETEARETH-4 PHOSPHATE, ALCOHOL DENAT, ORYZA SATIVA (RICE) STARCH, PENTAERYTHRITYL DISTEARATE, ALOE BARBADENSIS LEAF JUICE, FUCUS SERRATUS EXTRACT, TETRASELMIS SUECICA EXTRACT, LENS ESCULENTA (LENTIL) FRUIT EXTRACT, AMMONIUM ACRYLOYLDIMETHYLTAURATE/VP COPOLYMER, ETHYL OLEATE, TOTAROL, ACACIA SENEGAL GUM, PHENOXYETHANOL, PENTYLENE GLYCOL, PARFUM (FRAGRANCE), PALMITOYL PROLINE, CHLORPHENESIN, TETRASODIUM GLUTAMATE DIACETATE, TOCOPHEROL, ETHYL LINOLENATE, GLYCINE SOJA (SOYBEAN) OIL, XANTHAN GUM, ETHYLHEXYLGLYCERIN, MAGNESIUM PALMITOYL GLUTAMATE, SODIUM PALMITOYL SARCOSINATE, SODIUM PHYTATE, T-BUTYL ALCOHOL, PROPYL GALLATE, ALCOHOL
   Accordingly, after determining the therapeutic benefits desired, a serum solution 12, 14 or 16 is selected. Upon selection, the predetermined serum solution is applied as at 18 in FIG. 1. As also represented in FIG. 1, subsequent to the application of the predetermined serum solution 18 to the skin, a serum locking composition is applied, as at 20, in overlying, covering relation to the previously applied predetermined serum solution 18.

The serum locking composition 20 may in fact be applied in cream form and is infused with hydration-boosting hyaluronic acid and also incorporates shea and/or mangosteen butter with botanical extracts. Moreover, the serum locking composition 20 is “manually” worked in or infused into the predetermined serum solution 18 in order to facilitate adherence and encapsulation of a pre-heated wax composition 24, as set forth in greater detail hereinafter. As such, the serum locking composition 20 may be formulated to include the therapeutic ingredients as follows:

### Ingredients: Serum Locking Composition (Cream)

WATER (EAU/AQUA), HELIANTHUS ANNUUS (SUNFLOWER) SEED OIL, BEHENYL ALCOHOL, BUTYLENE GLYCOL, C10-18 TRIGLYCERIDES, GLYCERIN, BUTYROSPERMUM PARKII (SHEA) BUTTER, GARCINIA INDICA SEED BUTTER, CANDELILLA/JOJOBA/RICE BRAN POLYGLYCERYL-3 ESTERS, GLYCERYL STEARATE, CAPRYLIC/CAPRIC TRIGLYCERIDE, HYDROXYSTEARIC/LINOLENIC/OLEIC POLYGLYCERIDES, TREHALOSE, CETEARYL ALCOHOL, SODIUM STEAROYL LACTYLATE, PHENOXYETHANOL, FRAGRANCE (PARFUM), MICROCRYSTALLINE CELLULOSE, SODIUM BENZOATE, CHLORPHENESIN, ALOE BARBADENSIS LEAF JUICE, CITRIC ACID, SODIUM HYDROXIDE, TOCOPHEROL, BISABOLOL, LAMINARIA OCHROLEUCA EXTRACT, XIMENIA AMERICANA SEED OIL, BIOSACCHARIDE GUM-2, CELLULOSE GUM, SCLEROTIUM GUM, HYDROLYZED HYALURONIC ACID, XANTHAN GUM, PHYTIC ACID, SODIUM HYALURONATE, COMMIPHORA MUKUL RESIN EXTRACT, OENOTHERA BIENNIS (EVENING PRIMROSE) SEED EXTRACT, BRASSICA OLERACEA, ITALICA (BROCCOLI) EXTRACT, CAMELLIA SINENSIS LEAF EXTRACT, PUNICA GRANATUM FRUIT EXTRACT, SILYBUM MARIANUM EXTRACT, ROSMARINUS OFFICINALIS (ROSEMARY) LEAF EXTRACT

As set forth above, the embodiment of FIG. 1 emphasizes the heat amplification characteristics of the method and system 100 of the skin treatment. As such, a wax composition is pre-heated to an increased temperature, as at 22, sufficient to assume a substantially liquid state. Such a “liquid state” should facilitate the application of the pre-heated wax composition onto the skin, such as by utilizing an applicator brush or other appropriate instrument. Moreover, such a preferred pre-heated temperature of the wax composition may be generally about 122° F.-124° F. This temperature of the applied hot wax composition will also provide an overall comforting feeling to the skin.

It is noted that the precise temperature of the pre-heated wax composition 24, at the time of application to the skin, is not necessarily limited to these precise temperature parameters. Determinative factors should include a temperature of the wax composition sufficient to facilitate heat transfer to the skin of the user, while “encapsulating” the predetermined serum solution. However, the temperature of the wax composition should be sufficiently low to avoid discomfort to the skin. Additional features of the wax composition include its formulation to be substantially “non-porous” thereby eliminating or restricting the ability to remove hair from the skin, once applied, as at 1224.

Once pre-heated to a preferred temperature 22, the wax composition is applied to the skin, as at 24, in overlying, encapsulating relation to both the predetermined serum solution 18 and the serum locking composition 20. Further, the encapsulating characteristics of the wax composition is further defined by the pre-heated hot wax composition being allowed to cool, in place on the skin. Such cooling will have the effect of substantially solidifying the wax composition into a mask, as at 26. The mask 26 will extend in deliberately overlying, covering relation to the predetermined serum solution 18 and serum locking composition 20, as set forth above and represented in FIG. 1.

It should also be noted that the formation of the mask 26, upon cooling of the preheated wax composition will serve to encapsulate as well as “incubate” the underlying predetermined serum solution or booster. As a result, the beneficial properties of at least the underlying serum solution will be more thoroughly absorbed by the skin. Therefore, the formation of the mask from an at least partially solidified, film-like mask better accomplishes the “incubation” features. This further distinguishes the skin treatment of the present invention from conventional treatments by virtue of the fact that the wax mask may or may not necessarily have therapeutic ingredients formulated therein. However, one beneficial purpose of the wax mask is to perform the aforementioned encapsulation and incubation characteristics on the underlying predetermined serum solution, which in turn facilitates the absorption of the therapeutic and/or beneficial properties of the underlying serum solution into the skin.

Upon expiration of a predetermined period of time generally, but not exclusively, in the range of about 5-6 minutes, the formed mask 26 is removed, as at 28. The aforementioned solidification of the mask 26 will facilitate its concurrent removal in substantially one piece. Upon removal, the solidification and encapsulation of the wax composition into the mask 26 will serve to concurrently remove at least a majority of the previously applied predetermined serum solution 18 and overlying serum locking composition 20.

It is of further note that the time that the formed mask remains on the skin of the user, in encapsulating, incubating relation to the underlying predetermined serum solution and serum locking composition may be extended to generally about 10 minutes or even longer, thereby possibly resulting in increased therapeutic benefits. It being noted that categorization of the heat amplified skin treatment of the present invention in the “fast-facial” category may necessitate that a shorter period of time of the wax mask being left on the user's skin.

The preferred embodiment of FIGS. 2A-2B incorporates the embodiment of FIG. 1 and describes other procedures, techniques, therapeutic compositions, etc. which may be included in the method and system 100 of the heat amplified skin treatment. These described and represented additions further facilitate and enhance the desired results of the treatment, as presented in schematic form. Moreover, the embodiment of FIGS. 2A-2B represents implementation of the skin treatment at a treatment site which may be a customized and/or a specifically designed commercial site intended for the treatment of numerous users/customers.

With primary reference to FIG. 2A, initial preparation of the skin may involve the cleansing of the skin, as at 30, in order to remove any pre-existing compositions, materials, etc. Such pre-existing materials may include makeup, medicinal creams, ointments, etc. Such a cleansing process is facilitated by the utilization of one or more micellar wipes specifically structured and/or formulated to facilitate such cleansing procedures. In more specific terms, each of the plurality of micellar wipes are structured and formulated to remove makeup and other pre-existing materials without the use of additional water, while leaving the skin refreshed, smooth and at least minimally moist. As such, each of a plurality of micellar wipes may be formulated to include the following:

### Ingredients/Ingrédients: Micellar Wipes

WATER (EAU/AQUA), GLYCERIN, PROPYLENE GLYCOL, ERYTHRITOL, ALOE BARBADENSIS LEAF JUICE, PEG-40 HYDROGENATED CASTOR OIL, PEG-7 GLYCERYL COCOATE, PHENOXYETHANOL, ETHYLHEXYLGLYCERIN, FRAGRANCE (PARFUM)

Thereafter, a purifying gel-lotion cleanser is applied to the skin, preferably in mist form, as at 32. The cleansing lotion may be rubbed and/or massaged lightly into the various portions of the area of the skin. The purifying gel-lotion cleanser or “cleansing lotion” is infused with collagen and emollient humectants facilitating the removal of any remnants of pre-existing materials on the skin, while moisturizing the skin during the cleansing procedure. As such, a cleansing lotion or purifying gel-lotion cleanser may be formulated to include the following:

### INGREDIENTS: Cleansing Lotion

WATER (EAU/AQUA), PEG-7 GLYCERYL COCOATE, PROPANEDIOL, POLOXAMER 184, GLYCERIN, SQUALANE, XYLITYLGLUCOSIDE, ANHYDROXYLITOL, SOLUBLE COLLAGEN, AMMONIUM ACRYLOYLDIMETHYLTAURATE/VP COPOLYMER, PHENOXYETHANOL, CAPRYLIC/CAPRIC TRIGLYCERIDE, HYDROXYETHYL ACRYLATE/SODIUM ACRYLOYLDIMETHYL TAURATE COPOLYMER, ALLANTOIN, CHLORPHENESIN, FRAGRANCE (PARFUM), POLYSORBATE 60, T-BUTYL ALCOHOL, XYLITOL, SORBITAN ISOSTEARATE, GLUCOSE, BHT, BENZOIC ACID, SORBIC ACID

Thereafter, the cleansing lotion 32 is removed, as at 34, utilizing the aforementioned and described micellar wipes.

In order to further maintain a hydrated or moisturized condition of the skin, a mineral micro-mist toner is applied, preferably in mist form, as at 36. This toner solution is infused with mineral-rich water, is alcohol free and serves to invigorate the skin. As such, this toner solution 21 may be formulated to include the following therapeutic composition:

### Ingredients/Ingrédients: Toner

WATER (EAU/AQUA), GLYCERETH-26, PROPANDEIOL, SEA WATER, PEG-6 CAPRYLIC/CAPRIC GLYCERIDES, PHENOXYETHANOL, PENTYLENE GLYCOL, PPG-26-BUTETH-26, CHLORPHENESIN, PEG-40 HYDROGENATED CASTOR OIL, FRAGRANCE (PARFUM), CETRIMONIUM CHLORIDE, CITRIC ACID

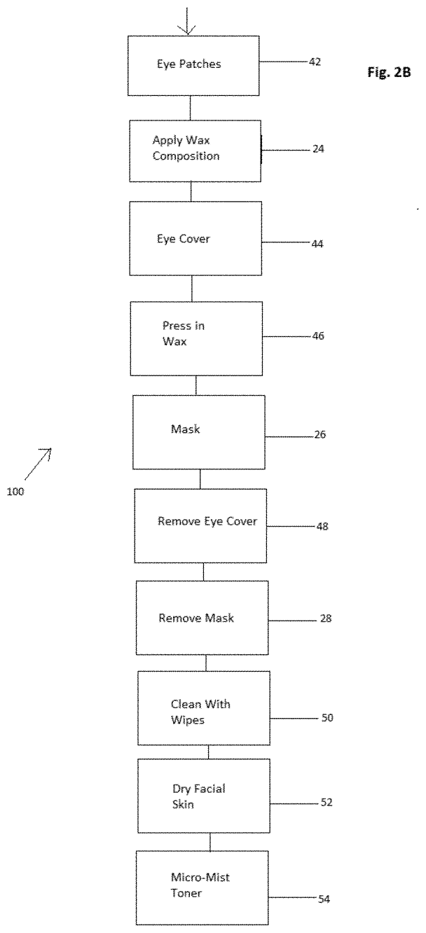

Subsequent to application of the toner solution 36, as set forth above, the skin is manually dried, as at 38, such as by “patting” until the skin is dry to the touch. After drying, as at 38, the selected, predetermined serum solution is applied to the skin, as at 18, preferably by hand, as also described with reference to the embodiment of FIG. 1. The application of the predetermined serum solution or booster 18 is followed by the application of the serum locking composition, as at 20 as represented in FIGS. 1 and 2A-2B. It is to be noted that the form of the serum locking composition may be applied as a cream. Further, the serum locking composition 20 may be initially applied to the hands of an individual performing the skin treatment on the user and lightly applied to the skin making sure that the serum locking composition 20 is well rubbed or “worked-in”. This will further enhance the adherence of the wax composition, as at 24, in FIGS. 1 and 2A-2B. In order to ensure a thorough and complete infusion or “working-in” of the serum locking composition 20 into the predetermined serum solution 18 and skin, an applying instrument, such as a roller, may also be utilized, as at 40. By way of example, an applying roller type instrument is generally indicated as 102 in FIG. 3. However, it is emphasized that while the roller type application instrument 102 is preferred, the infusion of the serum locking composition 20 into the skin and/or the predetermined serum solution 18, may be effectively accomplished either manually, without such an instrument 102 or utilizing any one of a possible variety of other instruments.

In addition and by way of example, when the treatment is applied to the face and in order to protect the delicate skin areas located immediately beneath the user's eyes, two eye patches (not shown) may be positioned, as at 42, in overlying relation to such underneath eye areas. This facilitates the protection of the facial skin beneath the eyes, such as during application of the pre-heated wax composition 24. The eye patches may assume a number of different sizes, configurations and structures. Further, the eye patches may be attached by means of a light adhesive or other attachment facility and may comprise a hyper-gel composition, which further protects the under eye area of the user's face.

Once the eye patches are properly positioned, as applicable, the pre-heated wax composition is applied to the area of the skin, as at 24, in covering, overlying and encapsulating relation to the previously applied predetermined serum solution 18 and serum locking composition 20, as also described with relation to the embodiment of FIG. 1. Also, when applied to the face, the wax composition is applied at 18, in overlying relation to the eye patches in a manner which avoids any potentially damaging contact with the eyes.

In cooperation therewith and subsequent to the application of the pre-heated wax composition to the face, as at 18, a heated eye cover 106, as represented in FIG. 5, may be applied in covering relation to the eyes of the user, as at 44 in FIG. 2B. The eye cover 106 may take a variety of different forms. In one such form and by way of example only, the structuring of the eye cover 106 may facilitate it being “automatically” heated once removed from its packaging and exposed to air. The automatically heated eye cover 106 will reach a temperature which is satisfying and soothing and will remain in place during the cooling of the pre-heated wax composition, which in turn will result in the formation of the at least partially solidified mask as at 26 and described in detail with reference to FIG. 1.

Yet additional procedures, techniques, etc. may include a manual pressing or “tapping” of the wax composition, as at 46, during the cooling thereof and the resulting formation of the mask, as at 26. This manual pressing or tapping facilitates the “encapsulation” of the wax composition and subsequent solidified mask, relative to the predetermined serum solution 18 and serum locking composition 20.

Upon expiration of a predetermined period of time generally, but not exclusively, in the range of about 5-6 minutes, the formed mask 26 is removed, as at 28. The aforementioned solidification of the mask 26 will facilitate its concurrent removal in substantially one piece. Upon removal, the solidification and encapsulation of the wax composition into the mask 26 will serve to concurrently remove at least a majority of the previously applied predetermined serum solution 18 and overlying serum locking composition 20. Since In situations where the pre-heated wax composition is applied to the skin, user's face, it will also be applied in overlying relation to the aforementioned eye patches, as applicable. As a result, the previously positioned eye patches will also be at least partially “encapsulated” and therefore be concurrently removed by and along with the facial wax mask, during its removal, as at 28.

Subsequent to the removal of the solidified mask, as at 28, the skin is thoroughly cleansed, utilizing one or more of the micellar wipes, as at 50. This step is performed in order to assure a cleansing and removal of all residues of the wax composition 24, predetermined serum solution 18 and serum locking composition 20, thereby avoiding any “sticky” and/or “tacky” feeling Thereafter, the user's skin is thoroughly dried, as at 52, utilizing any type of drying material or instrument.

One of the final steps in order to assure the comfort and appearance of the skin is the application of the aforementioned micro-mist toner, as at 54. The micro-mist toner or toner solution may be applied in mist form and includes a common formulation as that described with reference to procedure 36 as represented in FIG. 2A.

### Subject Parameters

| | Youth | Brightening | Clarifying |
|---|---|---|---|
| Product System | Women | Women | Women |
| | Age Group: 35-65 years | Age Group: 18-45 years | Age Group: 18-45 years |
| | Total number of subjects | Total number of subjects | Total number of subjects |
| | in completed study: 34 | in completed study: 32 | in completed study: 35 |
| Treatment + | Women | Women | Women |
| Product System | Age Group: 35-65 years | Age Group: 18-45 years | Age Group: 18-45 years |
| | Total number of subjects | Total number of subjects | Total number of subjects |
| | in completed study: 32 | in completed study: 32 | in completed study: 32 |

### Objectives

3-week and 6-week tests of both the product alone, and a regimen of treatment and product, and measured both consumer feedback and clinical measures.

| Test | | Objective |
|---|---|---|
| Treatment + | Youth | 6-week study (3 treatments) is designed to evaluate |
| Product System | Brightening | efficacy and consumer perception of facial treatment in |
| (3 Tests) | Clarifying | combination with product system |
| | | 3-week study (3 treatments) is designed to evaluate |
| | | efficacy and consumer perception of facial treatment in |
| | | combination with product system |
| Product System | Youth Product System | 3-week study) is designed to evaluate efficacy and |
| (3 tests) | Brightening Product | consumer perception of use of product system |
| | System | |
| | Clarifying Product System | |
| Facial Waxing + | Facial Waxing + | To evaluate the safety-in-use of a facial treatment after |
| Treatment | Treatment | getting waxed by a licensed esthetician in a panel of |
| (1 test) | (Youth, Brightening, | approximately 30 female subjects aged 18-65 years. |
| | Clarifying) | |
| Eye Mask Safety | Eye Mask Safety In-Use | 2-week safety-in-home to understand use. 50% were |
| In-Use | | contact lens wears, 25% had eyelash extensions. |
| (1 test) | | |

### Overview

Results across portfolio were dramatic and impressive

Cirefusion Therapie results are in the 65%-97% range for clinicals & 74%-100% range for consumer perception (Industry substantiates claims at 51% and considers claims “Strong” at 60%)

Youth 6-week study produced highest industry-leading results
Validated increased efficacy of repeated treatment & product usage
Clinical results scored 81%-94% across all four measures
Consumer results scored 100% of 5 of 6 measures (87% on 6th)

Brightening and Clarifying 3-week studies exceeded all expectations
Clinical results scored 65%-100% across seven measures
Consumer results scored 74%-100% on fourteen measures, with 12 above 90%
6-week tests for both Brightening and Clarifying in progress with results to be shared end of April

General Skincare Claims include:
Ophthalmologist-Tested
Dermatologist tested
Non-comedogenic
Does not clog pores
Safe for Contact Lens Wearers

Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents.

1. A heat amplified skin treatment method comprising:
applying one of a plurality of predetermined serum solutions, each having different therapeutic properties, to a user's skin,
formulating each of the plurality of predetermined serum solutions to include one of the therapeutic properties comprising anti-aging properties, brightening properties or clarifying properties;
pre-heating a wax composition into a liquid state,
applying the pre-heated wax composition directly to the skin in covering relation to the predetermined serum solution and in heat transferring relation to the skin,
forming a mask in encapsulating relation to the predetermined serum solution via a cooling and solidifying of the wax composition into a one piece configuration, while applied to the skin, and
removing the mask, in the one piece configuration, from the skin after a lapse of a predetermined period of time.

2. A heat amplified skin treatment method as recited in claim 1 comprising cleansing the skin with a purifying cleansing lotion prior to application of the one predetermined serum solution and removing the purifying cleansing lotion subsequent to cleansing.

3. A heat amplified skin treatment method as recited in claim 2 further comprising applying a moisturizing toner to the skin subsequent to the application of the purifying cleansing lotion and subsequently manually drying.

4. A heat amplified skin treatment as method recited in claim 1 comprising manually massaging a serum locking composition into the area of the skin in overlying, covering relation to the one predetermined serum solution, prior to application of the wax composition and formation of the mask.

5. A heat amplified skin treatment method as recited in claim 4 further comprising rolling the serum locking composition onto the skin, utilizing a rolling instrument, subsequent to the application thereof by massaging.

6. A heat amplified skin treatment method as recited in claim 1 further comprising manually pressing the wax composition into the skin for a predetermined period of time.

7. A heat amplified skin treatment method as recited in claim 1 further comprising cleansing the skin of wax composition and the one predetermined serum solution residue, subsequent to the removal of the mask.

8. A heat amplified skin treatment method as recited in claim 7 further comprising cleansing the skin of wax composition and a predetermined serum solution residue utilizing micellar wipes.

9. A heat amplified skin treatment method as recited in claim 7 further comprising applying a moisturizing toner mist to the skin subsequent to the cleansing of the wax composition and predetermined serum solution residue from the skin.

10. (canceled)

11. A heat amplified skin treatment method as recited in claim 1 wherein the one predetermined serum solution comprises the anti-aging properties primarily formulated to reduce appearance of lines/wrinkles and increase skin firmness.

12. A heat amplified skin treatment method as recited in claim 1 wherein the one predetermined serum solution comprises the brightening properties primarily formulated to improve skin tone, skin brightness and smoothness.

13. A heat amplified skin treatment method as recited in claim 1 wherein the one predetermined serum solution comprises clarifying properties primarily formulated to decrease oily skin (sebum), reduce appearance of pores and improve skin moisture.

14. A heat amplified skin treatment method as recited in claim 1 further comprising pre-heating the wax composition to a temperature of about 122° F.-124° F. and formulating the wax composition to provide an overall comforting feeling to the skin.

15. A system for a heat amplified skin treatment a user's skin, said system comprising:
one of a plurality of predetermined serum solutions applied to the skin,
said one predetermined serum solution formulated to include therapeutic properties comprising one of anti-aging properties, brightening properties or clarifying properties;
a wax composition applied directly to the skin in encapsulating relation to the predetermined serum solution,
said wax composition applied in a pre-heated, substantially liquid state, in heat transferring relation to the skin,
a mask at least partially defined by said wax composition being cooled and solidified on the skin in a one piece configuration, subsequent to application thereof, and
at least a majority of said mask comprising said one piece configuration and a substantially solid consistency, structured for substantially concurrent removal from the skin.

16. The system as recited in claim 15 wherein said wax composition comprises a non-porous configuration formulated to restrict hair removal from the skin.

17. The system as recited in claim 15 wherein said wax composition is pre-heated to a temperature of about 122° F.-124° F.

18. (canceled)

19. (canceled)

20. The system as recited in claim 15 wherein said one predetermined serum solution comprises said anti-aging properties primarily formulated to reduce appearance of lines/wrinkles and increases skin firmness.

21. The system as recited in claim 15 wherein said one predetermined serum solution comprises said brightening properties primarily formulated to improve skin tone, said brightening properties comprising skin brightness and smoothness.

22. The system as recited in claim 15 wherein said one predetermined serum solution comprises said clarifying properties primarily formulated to decrease oily skin (sebum) reduce appearance of pores and improve skin moisture.

23. The system as recited in claim 15 further comprising a serum locking composition applied to the skin in overlying, covering relation to said one predetermined serum solution and in underlying, encapsulated relation to said wax composition and said mask.

24. The system as recited in claim 23 wherein said serum locking composition comprises a skin soothing formulation comprising at least one of a shea butter and a mangosteen butter.

25. The system as recited in claim 15 further comprising a cleansing solution comprising infused collagen and emollient humectants, said cleansing solution formulated to moisturize and remove pre-existing compositions from the skin prior to application of said one predetermined serum solution.

26. The system as recited in claim 25 further comprising a plurality of cleansing wipes each formulated to remove compositions from the skin comprising residue of said wax composition and said one predetermined serum lotion subsequent to removal of said mask.

27. The system as recited in claim 26 further comprising a micro-mist toner solution including mineral rich water formulated to moisturize the skin, said micro-mist toner applied subsequent to removal of said mask after cleansing by said cleaning wipes.
